(19) United States
(12) Patent Application Publication
(10) Pub. No.: US 2016/0068527 A1
(43) Pub. Date: Mar. 10, 2016

(54) The Treatment of Inflammatory Disorders

(71) Applicant: CRESSET BIOMOLECULAR DISCOVERY LTD., Hertfordshire (GB)

(72) Inventors: Alan Leslie Rothaul, Oxfordshire (GB); Jeremy Gilbert Vinter, Hertfordshire (GB); Robert Arthur Scoffin, Cambridgeshire (GB)

(21) Appl. No.: 14/782,975

(22) PCT Filed: Apr. 9, 2014
(86) PCT No.: PCT/GB2014/051109

§ 371 Date: Oct. 7, 2015

(30) Foreign Application Priority Data
Apr. 9, 2013 (GB) 1306411.8
Apr. 9, 2013 (GB) 1306413.4

(51) Int. Cl. C07D 471/04; A61K 45/06; A61K 31/4375

(52) U.S. Cl. CPC C07D 471/04; A61K 31/4375; A61K 45/06

(57) ABSTRACT

The present invention provides Nalidixic acid and analogues of Nalidixic acid, and methods for treating inflammatory disorders. The Nalidixic acid and analogues of Nalidixic acid may be formulated for topical delivery or systemic delivery. The inflammatory disorders that may be treated with the Nalidixic acid and analogues of Nalidixic acid include respiratory diseases, chronic degenerative diseases, dematological conditions, chronic demyelinating diseases, dental diseases, ophthalmic conditions, inflammatory bowel diseases, and graft versus host diseases.

### FIELD OF THE INVENTION

This invention relates to the use of Nalidixic acid and analogues for the treatment of inflammatory disorders.

### BACKGROUND OF THE INVENTION

Immune-driven inflammatory events are a significant cause of many chronic inflammatory diseases where prolonged inflammation may cause tissue destruction and may result in extensive damage and eventual failure of the affected organ. In many cases the precise etiology of these diseases is unknown. Included in these diseases are the autoimmune diseases where, whilst the precise causative features of the disease are not understood, it is known that the inflammatory and tissue destructive aspects are the result of an inappropriate immune response directed at the body's own tissues. Conditions include those involving multiple organs, such as systemic lupus erythematosus (SLE) and scleroderma. Other types of autoimmune disease can involve specific tissues or organs such as the musculoskeletal tissue (e.g. rheumatoid arthritis, ankylosing spondylitis), the gastro-intestinal tract (e.g. Crohn's disease and ulcerative colitis), the central nervous system (e.g. Alzheimer's, multiple sclerosis, motor neurone disease, Parkinson's disease and chronic fatigue syndrome), pancreatic beta cells (e.g. insulin-dependent diabetes mellitus), the adrenal gland (e.g. Addison's disease), the kidney (e.g. Goodpasture's syndrome, IgA nephropathy, interstitial nephritis), exocrine glands (e.g. Sjogren's syndrome and autoimmune pancreatitis), the skin (e.g. psoriasis and atopic dermatitis) and the lung (e.g. asthma).

In addition, there are chronic inflammatory diseases whose etiology is to some extent identified. These may also exhibit extensive tissue/organ destruction and include conditions such as osteoarthritis, periodontal disease, diabetic nephropathy, chronic obstructive pulmonary disease, atherosclerosis, graft versus host disease, chronic pelvic inflammatory disease, endometriosis, chronic hepatitis and tuberculosis. These conditions are a major cause of illness in both the developed and developing world and in many cases are poorly treated by current therapies. For example, inflammation of skin structures (dermatitis) is a common set of conditions which include acne rosacea, acne vulgaris, allergic contact dermatitis, angioedema, atopic dermatitis, bullous pemiphigoid, cutaneous drug reactions, erythema multiforme, lupus erythrametosus, photodermatitis, psoriasis, psoriatic arthritis, scleroderma and urticaria. Diseases of the respiratory tract, for example asthma, chronic obstructive pulmonary disease (COPD) and Idiopathic Pulmonary Fibrosis (IPF) and inflammatory diseases of the gastrointestinal tract, such as Crohn's disease and ulcerative colitis, also represent a significant cause of illness in the population. These diseases are treated using a wide array of therapies, many of which have very severe side-effects.

Current treatments (if any) for inflammatory driven conditions include neutralizing antibodies, cytotoxics, corticosteroids, immunosuppressants, antihistamines and antimuscarinics. These treatments are often associated with inconvenient routes of administration and severe side-effects, leading to compliance issues.

There is a need for new effective and safe treatments.

Annexin-A1 (Lipocortin-1) is a 36 kDa protein which was first described in the late 1970's. It is found in many cell types and is known to play a key role in modulating the anti-inflammatory activity of exogenous and endogenous glucocorticosteroids. Annexin-A1 enhances the anti-inflammatory activity of steroids and in Annexin-A1 knock-out mice steroids are ineffective in animal inflammation models while Annexin-A1 itself is effective in animal models of inflammation (Ferretti M. and Dalli J. British Journal of Pharmacology (2009) 158, p936-946).

Inactive Annexin-A1 is released intracellularly by the nuclear action of glucocorticoid receptor stimulation. It is translocated to the cell membrane where it is phosphorylated by protein kinase C and released as an anti-inflammatory protein. The phosphatase PP2A is responsible for deactivating the anti-inflammatory activity of Annexin-A1 by direct de-phosphorylation and deactivation of protein kinase C (Yazid S. et al. Pharmacological Reports (2010) 62, p511-517). It is hypothesized that an inhibitor of PP2A would provide a potent anti-inflammatory agent.

### SUMMARY OF THE INVENTION

The present invention relates to the use of Nalidixic acid and analogues of Nalidixic acid in the treatment of inflammatory diseases.

Surprisingly it has been found that Nalidixic acid (I) and some analogues of Nalidixic acid are effective at treating inflammatory conditions.

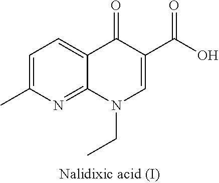 [SMILES: CCn1cc(C(=O)O)c(=O)c2ccc(C)nc21]

It has been found that Nalidixic acid and some analogues are potent inhibitors of the phosphatase PP2A thereby enhancing the anti-inflammatory activity of endogenous Annexin-A1. Nalidixic acid is an antibiotic most often used to treat urinary infections because it is rapidly excreted by the renal route and therefore has poor systemic pharmacokinetics. Typically this agent requires four times daily treatment by the oral route of administration to achieve anti-bacterial activity.

It has now been found that the use of Nalidixic acid or a Nalidixic acid analogue or a pharmaceutically acceptable salt thereof is effective in the treatment of inflammatory diseases such as, but not limited to those described above.

Thus, according to the present invention, Nalidixic acid and analogues of Nalidixic acid can be used for the treatment of inflammatory diseases.

### DESCRIPTION OF THE FIGURES

FIG. 1 represents the inhibition of % net histamine release from human mast cells by Nalidixic acid.

FIG. 2 represents the inhibition of Prostaglandin D2 release from human mast cells by Nalidixic acid.

FIG. 3 represents the release of Annexin-A1 from human mast cells in response to increasing concentrations of Nalidixic acid.

FIG. 4 represents the inhibition of ovalbumin induced BALF eosinophil count by Nalidixic acid given via the intranasal route in a murine model of asthma.

FIG. 5 represents the inhibition of the percentage of eosinophils of the total BALF cell count by Nalidixic acid in an ovalbumin induced murine model of asthma.

### DETAILED DESCRIPTION OF THE INVENTION

Administration of Nalidixic acid (I), or a pharmaceutically acceptable salt of Nalidixic acid is useful for the treatment of a range of inflammatory conditions.

According to another aspect of the present invention a compound of general formula (II)

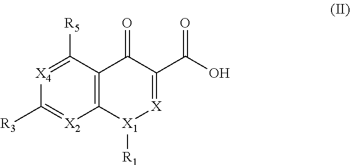

wherein,
X and X₁ independently represent CH or N;
X₂ represents C(R₂) or N;
X₄ represents C(R₄) or N;
R₁ is H, CF₃, CONH₂, CN, halogen, NH₂, NH-alkyl, alkyl, cycloalkyl or phenyl and is optionally substituted with one or more R₆; wherein R₁ may form part of a cycle with R₂;
R₂ is H, CF₃, CONH₂, CN, halogen, NH₂, alkyl, O-alkyl or S-alkyl; wherein R₂ may form part of a cycle with R₁, wherein the cycle is a 5-membered or 6-membered saturated or unsaturated cycle containing one or more atoms selected from C, N, S and O;
R₃ is H, CF₃, CONH₂, CN, halogen, NH₂, alkyl, O-alkyl, pyridyl, cycloalkyl or heterocycloalkyl and is optionally substituted with one or more R₆; wherein R₃ may form part of a cycle with R₄;
R₄ is H, F or O-alkyl; wherein R₄ may form part of a cycle with R₃, wherein the cycle is a 5-membered saturated or unsaturated cycle containing one or more atoms selected from C, N, S and O;
R₅ is H, F, CI, alkyl, O-alkyl or NH₂;
R₆ is F, alkyl, NH₂, NH-alkyl, CH₂NH₂ or OH;
or a pharmaceutically acceptable salt thereof, can be used for the treatment or prevention of an inflammatory condition.

Optionally, R₁, R₂ and R₃ are independently CF₃, CONH₂, CN, halogen or NH₂.

Alkyl refers to a linear or branched alkyl group having from 1 to 10 carbon atoms, preferably from 1 to 6 carbon atoms, more preferably, from 1 to 3 carbon atoms. Preferred examples of alkyl are methyl, ethyl, n-propyl and isopropyl.

Cycloalkyl refers to a saturated or partially saturated cyclic group of from 3 to 14 carbon atoms and no ring heteroatoms and having a single ring or multiple rings including fused, bridged, and spiro ring systems, wherein the cycloalkyl is optionally substituted by one or more substituents selected from CF₃, CONH₂, CN, halogen, NH₂, NH-alkyl, alkyl, cycloalkyl and phenyl.

Heterocycloalkyl refers to a saturated or partially saturated cyclic group having from 1 to 14 carbon atoms and from 1 to 6 heteroatoms selected from nitrogen, sulfur, or oxygen and includes single ring and multiple ring systems including fused, bridged, and spiro ring systems, wherein the cycloalkyl is optionally substituted by one or more substituents selected from CF₃, CONH₂, CN, halogen, NH₂, NH-alkyl, alkyl, cycloalkyl and phenyl. Preferred examples of heterocycloalkyl are piperidine, piperazine and pyrrolidine.

Embodiments of the invention that may be mentioned include those where cycloalkyl and/or heterocycloalkyl are unsubstituted.

It will be appreciated by those skilled in the art that reference herein to treatment extends to prophylaxis as well as the treatment of established conditions.

Compounds of formula (II) include some known quinolone antibiotics. Quinolone antibiotics are known to be broad spectrum antibiotics. They are chemotherapeutic bactericidal drugs and they work by preventing bacterial DNA from unwinding and duplicating. Known quinolone antibiotics include:
First-generation: cinoxacin, flumequine, oxolinic acid, piromidic acid, pipemidic acid, rosoxacin.
Second-generation: ciprofloxacin, enoxacin, fleroxacin, lomefloxacin, nadifloxacin, norfloxacin, ofloxacin, pefloxacin, rufloxacin.
Third-generation: balofloxacin, grepafloxacin, levofloxacin, pazufloxacin, sparfloxacin, temafloxacin, tosufloxacin.
Fourth-generation: clinafloxacin gatifloxacin, gemifloxacin, moxifloxacin, sitafloxacin, trovafloxacin, prulifloxacin.
In development: garenoxacin, delafloxacin.
Veterinary use: danofloxacin, difloxacin, enrofloxacin, ibafloxacin, marbofloxacin, orbifloxacin, sarafloxacin.

Compounds of formula (II) for use in the invention include (but are not limited to) known quinolone antibiotics as described above and novel compounds such as:

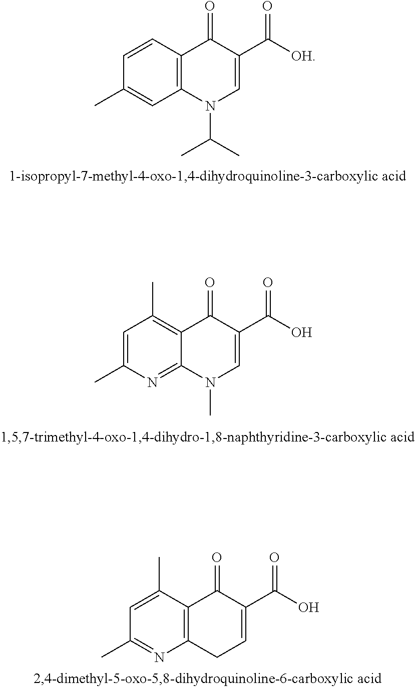 [SMILES: Cc1ccc2c(=O)c(C(=O)O)cn(C(C)C)c2c1 ; Cc1cc(C)c2c(=O)c(C(=O)O)cn(C)c2n1 ; Cc1cc(C)c2c(n1)CC=C(C(=O)O)C2=O]

It is understood that compounds for use in the invention include salts, e.g. sodium, potassium, ammonium, ethylenediamine, arginine, diethylamine, piperazine or N-methylglucamide salts, but also extends to metabolites and pro-drugs thereof. Most aptly the free acid or salt is employed.

Compounds for use in the invention, or their pharmaceutically acceptable salts, may be chiral, and it will be understood that this invention includes any diastereomers and enantiomers of formula (II). It will also be understood that the invention includes any isotopic derivatives of the compound of formula (I) and/or formula (II).

For the avoidance of doubt, compounds of formula (I) and (II) may contain the stated atoms in any of their natural or non-natural isotopic forms. In this respect, embodiments of the invention that may be mentioned include those in which:

a) the compound of formula (I) and/or formula (II) is not isotopically enriched or labelled with respect to any atoms of the compound; and

b) the compound of formula (I) and/or formula (II) is isotopically enriched or labelled with respect to one or more atoms of the compound.

References herein to an “isotopic derivative” relate to the second of these two embodiments. In particular embodiments of the invention, the compound of formula (I) and/or formula (II) is isotopically enriched or labelled (with respect to one or more atoms of the compound) with one or more stable isotopes. Thus, the compounds of the invention that may be mentioned include, for example, compounds of formula (I) and/or formula (II) that are isotopically enriched or labelled with one or more atoms such as deuterium or the like.

Preferred examples of compounds of formula (II) include cinoxacin, flumequine, oxolinic acid, piromidic acid, pipemidic acid and rosoxacin.

Nalidixic acid or the compounds of formula (II), or their pharmaceutically acceptable salts, according to the invention are used to treat inflammatory diseases including, but not exclusive to: autoimmune diseases involving multiple organs, such as systemic lupus erythematosus (SLE) and scleroderma, specific tissues or organs such as the musculoskeletal tissue (e.g. rheumatoid arthritis, ankylosing spondylitis), the gastro-intestinal tract (e.g. Crohn's disease and ulcerative colitis), the central nervous system (e.g. Alzheimer's, multiple sclerosis, motor neurone disease, Parkinson's disease and chronic fatigue syndrome), pancreatic beta cells (e.g. insulin-dependent diabetes mellitus), the adrenal gland (e.g. Addison's disease), the kidney (e.g. Goodpasture's syndrome, IgA nephropathy, interstitial nephritis) exocrine glands (e.g. Sjogren's syndrome and autoimmune pancreatitis), the skin (e.g. psoriasis and atopic dermatitis) and the lung (e.g. asthma); chronic inflammatory diseases such as osteoarthritis, periodontal disease, diabetic nephropathy, chronic obstructive pulmonary disease, atherosclerosis, graft versus host disease, chronic pelvic inflammatory disease, endometriosis, chronic hepatitis and tuberculosis; IgE mediated (Type I) hypersensitivities such as rhinitis, asthma, anaphylaxis and dermatitis. Dermatitis conditions include actinic keratosis, acne rosacea, acne vulgaris, allergic contact dermatitis, angioedema, atopic dermatitis, bullous pemiphigoid, cutaneous drug reactions, erythema multiforme, lupus erythrametosus, photodermatitis, psoriasis, psoriatic arthritis, scleroderma and urticaria. Conditions of the eye, such as diabetic retinopathy, macular degeneration, choroidal neovascular membrane, cystoid macularedema, epi-retinal membrane, macular hole, dry eye, uveitis and conjunctivitis, may also be treated. In particular, Nalidixic acid or the analogues of formula (II), or their pharmaceutically acceptable salts, are used to treat chronic degenerative disease such as rheumatoid arthritis, osteoarthritis or osteoporosis; chronic demyelinating disease such as multiple sclerosis; respiratory disease such as asthma or chronic obstructive pulmonary disease; inflammatory bowel disease such as ulcerative colitis or Crohn's disease; dermatological conditions such as psoriasis, scleroderma or atopic dermatitis; dental disease such as periodontal disease or gingivitis; diabetic nephropathy; lupus nephritis; IgA nephropathy; glomerulonephritis; systemic lupus erythematosus; graft versus host disease; ophthalmic conditions including age related macular degeneration, conjunctivitis, diabetic retinopathy, choroidal neovascular membrane, cystoid macular edema, epi-retinal membrane, macular hole, dry eye or uveitis.

It may be advantageous to use Nalidixic acid or an analogue of Nalidixic acid by topical administration, in the treatment of inflammatory diseases. When used topically, the compounds of the invention are used to treat inflammatory diseases including, but not exclusive to: autoimmune diseases involving specific tissues or organs such as the gastro-intestinal tract (e.g. Crohn's disease and ulcerative colitis), the skin (e.g. psoriasis and atopic dermatitis) and the lung; chronic inflammatory diseases such as chronic obstructive pulmonary disease and hypersensitivities such as rhinitis, asthma, anaphylaxis and dermatitis. Dermatitis conditions include actinic keratosis, acne rosacea, acne vulgaris, allergic contact dermatitis, angioedema, atopic dermatitis, bullous pemiphigoid, cutaneous drug reactions, erythema multiforme, lupus erythrametosus, photodermatitis, psoriasis, psoriatic arthritis, scleroderma and urticaria. In particular the compounds of the invention are used to treat respiratory disease such as asthma or chronic obstructive pulmonary disease; inflammatory bowel disease such as ulcerative colitis or Crohn's disease; dermatological conditions such as psoriasis, scleroderma or atopic dermatitis and dental diseases such as gingivitis and periodontal disease.

Nalidixic acid or compounds of formula (II) or salts thereof may be used according to the invention when the patient is also administered another therapeutic agent or wherein the compounds of the invention are provided in combination with another therapeutic agent, wherein the therapeutic agent is selected from corticosteroids (examples including Cortisol, cortisone, hydrocortisone, dihydrocortisone, fludrocortisone, prednisone, 35prednisolone, deflazacort, flunisolide, beconase, methylprednisolone, triamcinolone, betamethasone, and dexamethasone), cytotoxics, disease modifying anti-rheumatic drugs (DMARDs) (examples including azulfidine, aurothiomalate, bucillamine, chlorambucil, cyclophosphamide, leflunomide, methotrexate, mizoribine, penicillamine and sulphasalazine), immunosuppressants (examples including azathioprine, cyclosporin, mycophenolate), COX inhibitors (examples including aceclofenac, acemetacin, alcofenac, alminoprofen, aloxipirin, amfenac, aminophenazone, antraphenine, aspirin, azapropazone, benorilate, benoxaprofen, benzydamine, butibufen, celecoxib, chlorthenoxacine, choline salicylate, chlometacin, dexketoprofen, diclofenac, diflunisal, emorfazone, epirizole, etodolac, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, mefenamic acid, metamizole, mofebutazone, mofezolac, nabumetone, naproxen, nifenazone, oxametacin, phenacetin, pipebuzone, pranoprofen, propyphenazone, proquazone, rofecoxib, salicylamide, salsalate, sulindac, suprofen, tiaramide, tinoridine, tolfenamic acid, zomepirac) neutralizing antibodies (examples including etanercept and infliximab), antibiotics (examples including doxycycline and minocycline).

It will often be advantageous to use Nalidixic acid or compounds of formula (II), or a pharmaceutically acceptable salt thereof, in combination with drugs used for pain therapy. Such another drug may be an opiate or a non-opiate such as baclofen or a neuropathic pain agent such as gabapentin. Other compounds that may be used include: a non-steroidal anti-inflammatory drug (e.g. acetaminophen, acetylsalicyclic acid, naproxen), a narcotic analgesic, a local anesthetic, an NMDA antagonist, a neuroleptic agent, an anti-convulsant, an anti-spasmodic, an antidepressant or a muscle relaxant.

The anti-inflammatory activity of a compound of the invention can be characterized in appropriate in vitro or in vivo assays as described in the examples. Histamine (Example 1) and PGD2 (Example 2) released from IgE challenged human mast cells are both inhibited by Nalidixic acid treatment in a dose related manner. In addition the release of Annexin-A1 (Example 3) is increased by treatment with Nalidixic acid in a dose related manner.

The anti-inflammatory activity of the compounds of the present invention is not linked to their anti-bacterial activity and their anti-inflammatory effect can be observed at non anti-bacterial concentrations of Nalidixic acid or analogues of formula (II). Thus, according to another aspect of the invention, Nalidixic acid (I) or analogues of formula (II) or a pharmaceutically acceptable salt thereof can be used in the treatment or prevention of inflammatory conditions when the amount, dose or concentration of Nalidixic acid or analogue or salt thereof has no substantial antibiotic activity. In circumstances in which bacterial infection does not represent a component of the disease, the use of Nalidixic acid or analogue of formula (II) or a pharmaceutically acceptable salt thereof at sub-antibiotic doses would avoid unnecessary exposure to antibacterial activity that may lead to the generation of bacterial resistance.

According to an additional aspect of the invention Nalidixic acid or a compound of formula (II) or a pharmaceutically acceptable salt thereof can be used to potentiate the anti-inflammatory action of glucocorticosteroids. This activity has been demonstrated by the use of the appropriate in vitro and in vivo assays. Thus the use of a compound of the invention with steroids allows the use of traditionally sub-therapeutic, and therefore non-harmful, doses of steroids with greatly potentiated anti-inflammatory activity. Nalidixic acid or the compounds of formula (II), or their pharmaceutically acceptable salts, may be used according to the invention when the patient is also administered one or more glucocorticosteroids or wherein the compounds of the invention are provided in combination with one or more glucocorticosteroids. Glucocorticosteroids which can be used in the invention include, but are not limited to, beclomethasone, betamethasone, budesonide, cortisone, dexamethasone, hydrocortisone, fluticasone, meprednisone, mometasone, paramethasone and prednisolone. Particularly preferred is the use in combination with dexamethasone, prednisolone, cortisone, dihydrocortisone and hydrocortisone.

The compounds of the invention can be used as an anti-inflammatory agent to treat inflammatory conditions. In some instances, the diseases described above may be accompanied by a microbial infection. Such infection may be fungal, viral or bacterial. For example exacerbations of both asthma and COPD may be associated with concurrent infections of the lung. Dermal inflammatory diseases such as atopic dermatitis can also be associated with localised infection. The compounds of the present invention can be used to treat inflammation in the presence or absence of a microbial infection. When a microbial infection is present, the compounds of the invention may be used when the patient is also administered antibiotics or the compounds of the invention are administered in combination with antibiotics.

The compounds described herein may be formulated for administration in any convenient way, and the invention therefore also includes pharmaceutical compositions comprising Nalidixic acid or an analogue of formula (II) or pharmaceutically acceptable salts thereof together, if desirable, in admixture with one or more pharmaceutically acceptable diluents or carriers.

Any suitable route of administration can be used. For example, any of oral, topical, parenteral, ocular, rectal, vaginal, inhalation, buccal, sublingual and intranasal delivery routes may be suitable.

Pharmaceutical compositions suitable for topical administration are preferred. Any suitable route of administration for topical delivery of the active agent to the site of the disease can be used. Compositions suitable for topical administration to the lungs, the skin or the gastrointestinal tract are particularly preferred. Examples of various types of preparation for topical administration include ointments, lotions, creams, gels, foams, sprays, aerosols, powders, capsules or cartridges for use in an inhaler or insufflator, drops, solutions/suspensions for nebulization, suppositories, retention enemas, and pessaries.

Pharmaceutical compositions of Nalidixic acid or an analogue of formula (II) or a pharmaceutical salt thereof, suitable for systemic administration, represent another aspect of the invention. Oral pharmaceutical compositions may be preferred. Examples of various types of preparation for oral administration include capsules, tablets, pellets and liquid compositions. Parenteral compositions may also be preferred.

The dose of the active agent will depend on the nature and degree of the condition, the age and condition of the patient and other factors known to those skilled in the art. A typical dose is from 0.1 to 100 mg, for example, 10 to 100 mg of the active ingredient dependent upon the type of preparation involved. Preferably the dose is given one to three times per day. The dose of the active agent in the compositions of the invention can have antibiotic activity or it can be a sub-antibiotic dose. Preferably the active agent is present in an amount that has no substantial antibiotic activity. Antibiotic activity means that the concentration of Nalidixic acid or an analogue of Formula (II) would not have clinically relevant activity on the growth of pathogenic bacteria involved in infectious conditions. For susceptible bacterial strains this would be approximately less than 1 μg/ml

The compositions may further comprise one or more steroids and/or another therapeutic agent. Typically, a composition comprising Nalidixic acid or a compound of formula (II) or a pharmaceutically acceptable salt thereof and one or more steroids will comprise the steroid(s) in a range of 0.001% to 5% wt/wt of the formulation. Preferably the steroid is present in a normally sub-therapeutic dose of less than 1% wt/wt of the formulation, due to the synergistic effect of the compounds of the invention as described above, although the specific dose will depend on the particular steroid used. For example, when Nalidixic acid is used, it is present within the compositions in the range of 0.001% to 5% wt/wt of the formulation and the steroid is present in a therapeutic dose of less than 1% wt/wt of the formulation.

Nalidixic acid is generally prepared through a multi-step synthetic route, which lends itself to several modifications which allow for the synthesis of Nalidixic acid analogues, such as those of formula (II):

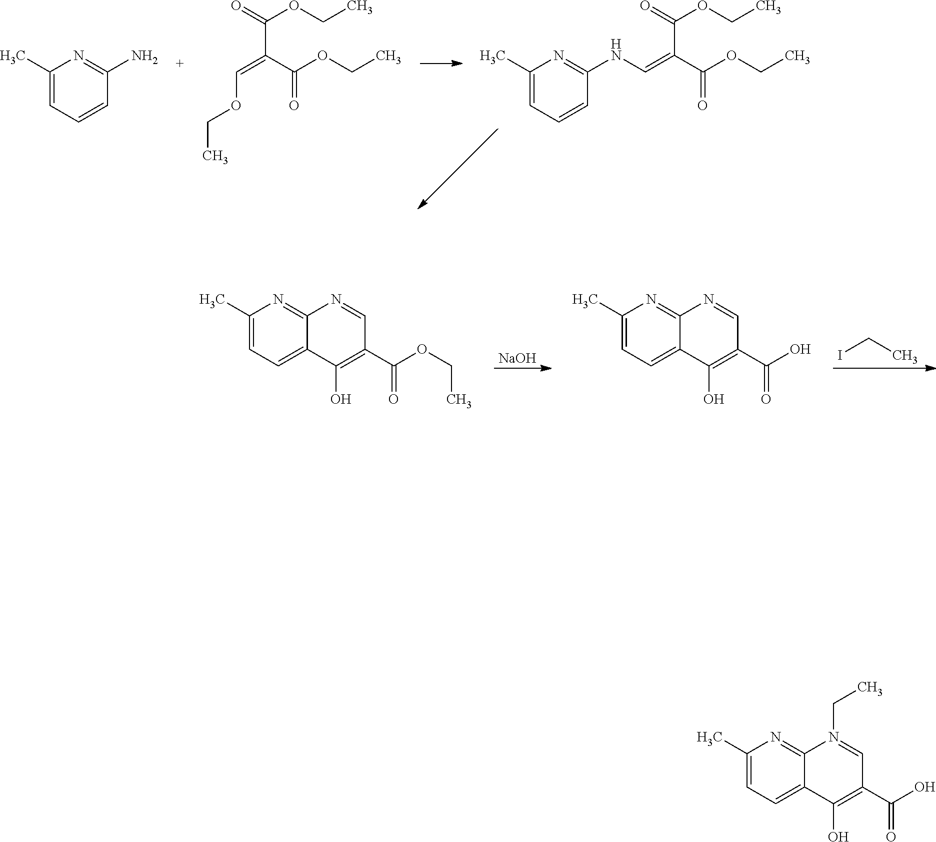

Nalidixic acid analogues of formula (II) for use in the invention may be prepared by a multi-step synthetic procedure, as shown in the following Scheme.

The synthesis proceeds by a cyclisation starting from a di-substituted benzene compound of general formula (III):

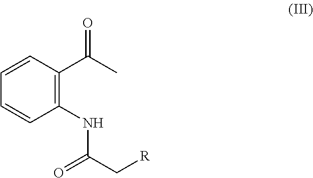

wherein R is any suitable group known to the skilled person.

The starting material is then cyclized through a Camps cyclisation to give compounds of general formula (IIIa) and (IIIb)

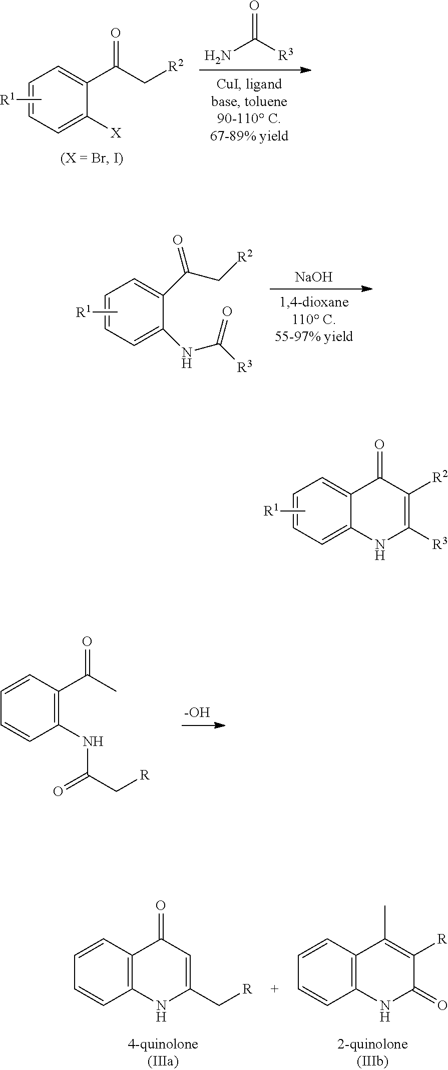

The 4-quinolone derivative of formula (IIIa) can then be isolated and further reacted to form 4-quinolone derivatives such as:

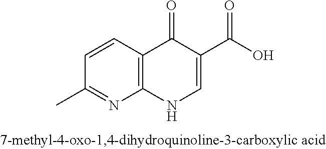

The anti-inflammatory activity of the compounds of formula (II), or their pharmaceutically acceptable salts, can be determined by assessing their capability of inhibiting the release of histamine or PDG₂ from Human Mast Cells or promoting the release of Annexin-A1.

The following Assays illustrate the invention:

### Example 1

### The Inhibition of Histamine Release from Human Mast Cells by Nalidixic Acid

Protocol: Human derived cord mast cells were cultured using the following method.

Commercially available CD34⁺ stem cells were cultured for 2 weeks in StemSpan (StemCell Technologies, Grenoble, France) serum-free medium supplemented with 100 ng/ml human SCF, 50 ng/ml IL-6 and 1 ng/ml IL-3, and 100 μg/ml penicillin/streptomycin (Peprotech, London, UK). After eight weeks, cells were cultured in StemSpan with 10% FCS. The cells were passaged into new medium every week. Cells were used for experiments between 11 and 18 weeks following confirmation by microscopic examination, c-kit and FcRε1 staining (by FACS), of mast cell morphology. For assessment of drug effects, Nalidixic Acid was incubated for 5 min with aliquots of 2×10⁵ CDMCs (cord derived mast cells) cultured in 10% FCS medium.

### Measurement of Histamine Release

A commercially-available enzyme immunoassay was used to detect and quantify histamine released in the supernatant (SPI bio, Strasbourg, France). The assay was conducted following the manufacturer protocols. A standard curve ranging from 0.39-50 nM histamine was prepared using the reagent provided and the optical density was then read within 60 min in a microplate reader (at 405 nm). In some cases, the total cell content of histamine was established by freeze thawing of cells prior to challenge

The results from these experiments are shown in FIG. 1. The data clearly demonstrates a dose related inhibition of the inflammatory mediator histamine by Nalidixic acid.

### Example 2

### Inhibition of Prostaglandin D₂ Release Form Human Mast Cells by Nalidixic Acid

Human cord derived mast cells were cultured using the methodology described in Example 1.

### Measurement of PGD₂ Release

A commercially-available enzyme immunoassay (Cayman Chemical, Michigan, USA) was used to detect and quantify PGD₂ released in the supernatant. The assay was conducted following the manufacturer protocols. A standard curve ranging from 78-10,000 pg/ml PGD₂ was prepared using the reagent provided and the optical density was then read within 60 min in a microplate reader (at 405 nm).

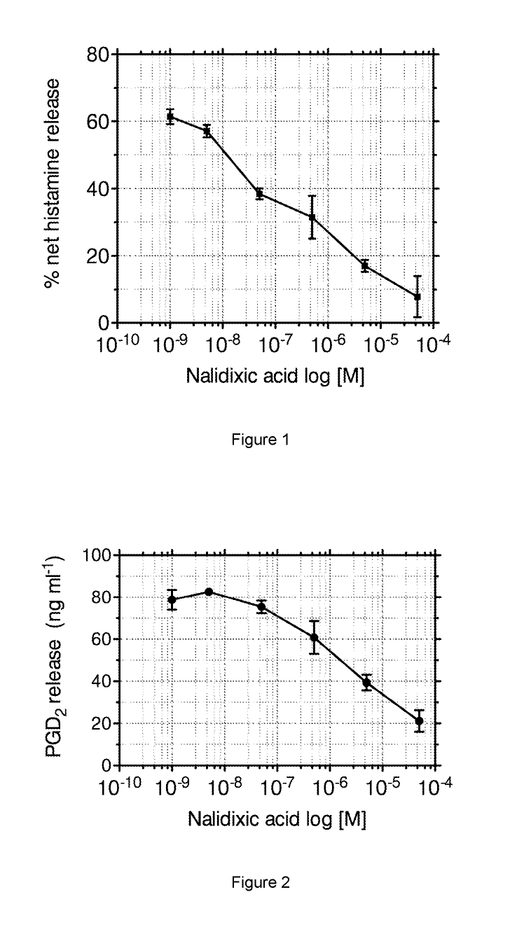

The results from these experiments are shown in FIG. 2. The data illustrates a dose related inhibition by Nalidixic acid of the inflammatory prostanoid PGD₂

### Example 3

### Nalidixic Acid Promotes the Release of Annexin-A1 from Human Mast Cells

Human cord derived mast cells were cultured using the methodology described in Example 1.

Anx-A1 protein levels in conditioned medium were determined by ELISA. Briefly, 96-well flat-bottomed ELISA plates (Greiner, Gloucestershire, UK) were coated with 1 μg anti-Anx-A1 mAb 1B in bicarbonate buffer (pH 9.6) and incubated overnight at 4° C. After washing in the bicarbonate buffer, potentially uncoated sites were blocked with 100 μL of PBS containing 1% BSA for 1h at room temperature. Sample aliquots (100 μL) or Anx-A1 standard solutions (prepared in 0.1% Tween-20 in PBS; concentration ranging between 10 and 0.001 μg/mL) were added for 1h at 37° C. After extensive washing in PBS/Tween-20, 100 μL of a polyclonal rabbit anti-human Anx-A1 serum (Zymed, Invitrogen, Paisley, UK; diluted 1:1000 in PBS/Tween-20) was added (1h at 37° C.) prior to incubation with donkey anti-rabbit 1 gG conjugated to alkaline phosphatase (1:1000; Sigma). The colour was developed by addition of 100 μL p-nitrophenyl phosphate (1 mg/mL in bicarbonate buffer, pH 9.6). Absorbance was read at 405 nm (with a 620-nm reference filter) in a microplate reader (Titertek™, Vienna, Austria). Anx-A1 levels in the study samples were read against the standard curve and expressed as ng/ml.

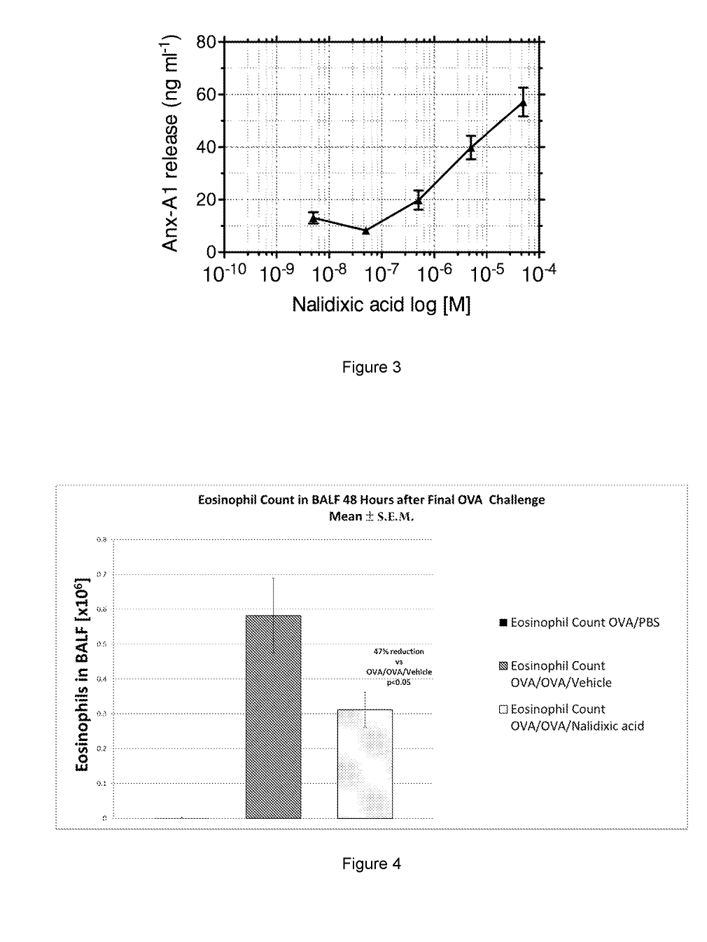

The results, as shown in FIG. 3, highlight the increase of the anti-inflammatory Annexin-A1 released from human mast cells in response to increasing concentrations of Nalidixic acid.

### Example 4

### Nalidixic Acid Given Topically to the Lung in a Murine Model of Asthma

The effects of locally administered Nalidixic acid were studied in a mouse model of asthma. Female BALB/c mice, 8 animals per group, were sensitised to ovalbumin (OVA, 10 mg) by intra-peritoneal injection on days 1 and 14 of the study. On days 20, 21, 22 and 23 animals either received a challenge dose of ovalbumin (50 μg) to the lung given by intranasal administration or 50 μl of phosphate buffered saline (PBS, non-challenged group). Animals challenged with OVA also received either PBS vehicle or Nalidixic acid (0.3 mg/kg of body weight) given by intranasal administration for inhalation into the lung. This was administered 30 minutes prior to and 6 hours post each ovalbumin challenge on days 21 to 23 and in addition on day 24, the day between the last challenge and the terminal day of the experiment. Forty-eight hours after the final OVA challenge, mice were anaesthetised with a combination of ketamine hydrochloride (Narketan, 2 mg/mouse) and xylazine hydrochloride (Rompun, 0.07 mg/mice) and sacrificed by carotid exsanguination.

Bronchoalveolar lavage fluid (BAL) was obtained as follows. The trachea was cannulated. Phosphate Buffered Saline (PBS) was used as the lavage fluid, 3 volumes (0.4 ml; 0.3 ml; 0.3 ml; total 1 mL) were gently instilled and withdrawn on 3 consecutive occasions using a 1 ml BD syringe and then placed in an Eppendorf tube (˜1 ml).

Batches of these were next centrifuged in a desktop Eppendorf centrifuge (5 min/3500 rpm/4° C.). Supernatants were removed and cell plugs re-suspended in 600 μl of PBS by vigorously shaking closed tubes content on Vortex. 300 μl of each suspension was then removed and placed in the marked sample chamber. Filter cards (Shandon Filter Cards, brown, for use with samples 0.4 ml or less, ref#5991023) are pre-assembled between sample chamber and a marked microscope slide (Thermo Scientific, Shandon Cytoslide, coated microscope slides, ref#5991056), fastened together with a clip and placed in the upright position inside the centrifuge in the designated position.

Cellular counts from the obtained BALF on a haematological analyser (Sysmex XT-2000iV). Statistical comparison of BALF cellular counts was performed using a Mann Whitney test.

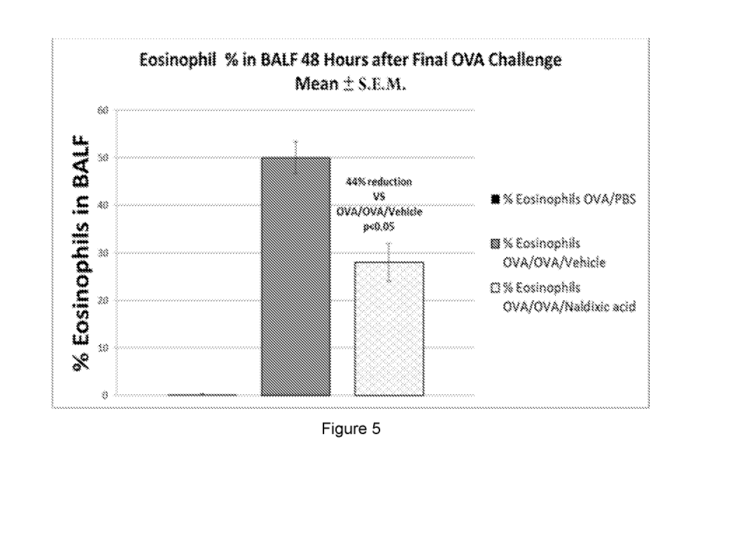

A key aspect of this model is an assessment of eosinophil recruitment into BALF induced by the immunological response to ovalbumin sensitisation. As expected, sensitised animals when challenged with OVA and given PBS intranasal (OVA/OVA/Vehicle) demonstrated a marked increase in eosinophil count in BALF compared with animals that were not challenged (OVA/PBS). However, intranasal administration of Nalidixic acid (OVA/OVA/Nalidixic acid) resulted in a marked and statistically significant (p<0.05) 47% decrease in total BALF eosinophil counts compared with challenged vehicle treated animals (OVA/OVA/Vehicle) (FIG. 4). In addition the percentage of eosinophils of the total cellular count was also markedly and statistically significantly (p<0.05) reduced by 44% in the Nalidixic acid versus vehicle treated group (FIG. 5).

These data demonstrate the efficacy of Nalidixic acid when given topically to the lung in model of asthma and indicate its potential as a treatment for this condition when given by oral inhalation.

1. A method for treating or preventing an inflammatory condition, the method comprising administering to a patient at least one of a Nalidixic acid of formula (I) or an analogue or a pharmaceutically acceptable salt thereof:

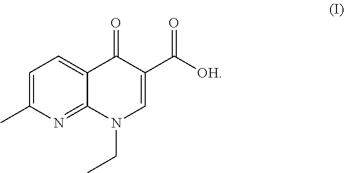 [SMILES: CCn1cc(C(=O)O)c(=O)c2ccc(C)nc21]

2. The method according to claim 1, wherein the inflammatory condition is a respiratory disease, a chronic degenerative disease, a dermatological condition, a chronic demyelinating disease, an inflammatory bowel disease, a dental disease, a graft versus host disease, or an ophthalmic condition.

3. The method according to claim 1, wherein the Nalidixic acid, the analogue, or the pharmaceutically acceptable salt thereof is formulated for topical delivery.

4. The method according to claim wherein the Nalidixic acid, the analogue, or the pharmaceutically acceptable salt thereof is formulated for topical delivery to at least one of the skin, the lungs, or the gastrointestinal tract.

5. The method according to claim 1, wherein the Nalidixic acid, the analogue, or the pharmaceutically acceptable salt thereof is formulated for topical delivery to the skin and the condition is a dermatological condition.

6. The method according to claim 1, wherein the Nalidixic acid, the analogue, or the pharmaceutically acceptable salt thereof is formulated for topical delivery to the lung and the condition is a lung condition.

7. The method according to claim 1, wherein the Nalidixic acid, the analogue, or the pharmaceutically acceptable salt thereof is formulated for topical delivery to the gastrointestinal tract and the condition is an inflammatory bowel disease.

8. The method according to claim 1, wherein the condition is a dental disease.

9. The method according to claim 1, wherein the Nalidixic acid, the analogue, or the pharmaceutically acceptable salt thereof is formulated for systemic delivery.

10. The method according to claim 1, further comprising administering one or more glucocorticosteroids to the patient.

11. The method according to claim 1, further comprising administering to the patient at least one therapeutic agent selected from the group consisting of angiostatic peptides; angiostatic steroids; modulators of VEGF or FGF; non-steroidal anti-inflammatory drugs (NSAIDs) formulated for ocular use; glucocorticosteroids; leukotriene modulators; anti-histamines; and general cytokine/growth factor modulating agents.

12. The method according to claim 10, wherein [i] the Nalidixic acid, the analogue, or the pharmaceutically acceptable salt thereof, and [ii] the one or more glucocorticosteroids are administered in combination.

13. The method according to claim 1, wherein the Nalidixic acid of formula (I) or the analogue or the pharmaceutically acceptable salt thereof is part of a pharmaceutical composition.

14. The method according to claim 13, wherein the pharmaceutical composition is suitable for topical delivery.

15. The method according to claim 13, wherein the pharmaceutical composition comprises the Nalidixic acid or the pharmaceutically acceptable salt thereof.

16. The method according to claim 1, wherein the Nalidixic acid analogue is a compound of formula (II): 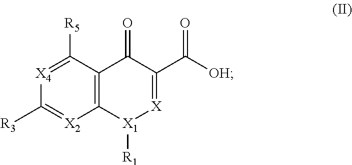
wherein,
X and X₁ independently represent CH or N;
X₂ represents C(R₂) or N;
X₄ represents C(R₄) or N;
R₁ is H, CF₃, CONH₂, CN, halogen, NH₂, NH-alkyl, alkyl, cycloalkyl, or phenyl, and is optionally substituted with one or more R₆; wherein R₁ may form part of a cycle with R₂;
R₂ is H, CF₃, CONH₂, CN, halogen, NH₂, alkyl, O-alkyl, or S-alkyl; wherein R₂ may form part of a cycle with R₁;
R₃ is H, CF₃, CONH₂, CN, halogen, NH₂, alkyl, O-alkyl, pyridyl, cycloalkyl, or heterocycloalkyl and is optionally substituted with one or more R₆; wherein R₃ may form part of a cycle with R₄;
R₄ is H, F, or O-alkyl; wherein R₄ may form part of a cycle with R₃;
R₅ is H, F, Cl, alkyl, O-alkyl, or NH₂;
R₆ is F, alkyl, NH₂, NH-alkyl, CH₂NH₂, or OH;
or a pharmaceutically acceptable salt thereof.

17. (canceled)

18. The method according to claim 1, wherein an amount of the compound of formula (I) or the pharmaceutically acceptable salt thereof that is administered to the patient has no substantial antibacterial activity.

19. The method according to claim 11, wherein [i] the Nalidixic acid, the analogue, or the pharmaceutically acceptable salt thereof, and [ii] the at least one therapeutic agent are administered in combination.

20. The method according to claim 16, wherein an amount of the compound of formula (II) or a pharmaceutically acceptable salt thereof that is administered to the patient has no substantial antibacterial activity.

21. A Nalidixic acid analogue according to formula (II): 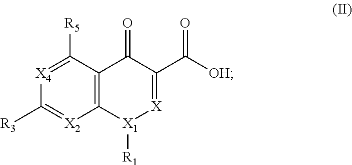
wherein,
X and X₁ independently represent CH or N;
X₂ represents C(R₂) or N;
X₄ represents C(R₄) or N;
R₁ is H, CF₃, CONH₂, CN, halogen, NH₂, NH-alkyl, alkyl, cycloalkyl, or phenyl, and is optionally substituted with one or more R₆; wherein R₁ may form part of a cycle with R₂;
R₂ is H, CF₃, CONH₂, CN, halogen, NH₂, alkyl, O-alkyl, or S-alkyl; wherein R₂ may form part of a cycle with R₁;
R₃ is H, CF₃, CONH₂, CN, halogen, NH₂, alkyl, O-alkyl, pyridyl, cycloalkyl, or heterocycloalkyl, and is optionally substituted with one or more R₆; wherein R₃ may form part of a cycle with R₄;
R₄ is H, F, or O-alkyl; wherein R₄ may form part of a cycle with R₃;
R₅ is H, F, Cl, alkyl, O-alkyl, or NH₂;
R₆ is F, alkyl, NH₂, NH-alkyl, CH₂NH₂, or OH;
or a pharmaceutically acceptable salt thereof.

22. A pharmaceutical composition comprising:
at least one of Nalidixic acid, a pharmaceutically acceptable salt of Nalidixic acid, or the Nalidixic acid analogue according to claim 21; and
at least one of a diluent or a carrier.
